(19) United States
(12) Patent Application Publication
(10) Pub. No.: US 2010/0129835 A1
(43) Pub. Date: May 27, 2010

(54) Methods for Selecting Improved Strains

(76) Inventor: Elizabeth A. Bodie, San Carlos, CA (US)

Correspondence Address: DANISCO US INC.;ATTENTION: LEGAL DEPARTMENT, 925 PAGE MILL ROAD, PALO ALTO, CA, 94304, US

(21) Appl. No.: 12/522,671

(22) PCT Filed: Jan. 8, 2008
(86) PCT No.: PCT/US08/00311

§ 371 Date: Jan. 27, 2010

Related U.S. Application Data
(60) Provisional application No. 60/879,942, filed on Jan. 10, 2007.

(51) Int. Cl. G01N 33/53

(52) U.S. Cl. 435/7.31

(57) ABSTRACT

The present invention relates to a system for improving a strain of filamentous fungus that is useful in producing an enzyme of interest such as a cellulase. The invention involves generating genetic diversity in a strain of fungal cells, subjecting the population of cells to selection, and isolating the cells that overproduce the enzyme. The selection techniques involve culturing the population of cells in purified cellulose and/or at an elevated temperature that is adverse to production of the enzyme.

### CROSS-REFERENCES TO RELATED APPLICATION

The present application claims benefit of and priority to U.S. Provisional Application Ser. No. 60/879,942, entitled “METHODS FOR SELECTING IMPROVED STRAINS”, filed Jan. 10, 2007, incorporated herein by reference in its entirety.

### 1. GOVERNMENT SUPPORT

Portions of this work were funded by Subcontract No. ZCO-30017-01 with the National Renewable Energy Laboratory under Prime Contract No. DE-AC36-99GO10337 with the U.S. Department of Energy. Accordingly, the United States Government may have certain rights in this invention.

### 2. INTRODUCTION

The invention relates to methods for selecting improved fungal strains for enzyme production.

### 3. BACKGROUND OF THE INVENTION

Biomass which largely consists of cellulose, hemicellulose and lignin has attracted increasing attention as an important renewable source of energy (including nutritional energy). The amount of carbon fixed by photosynthesis has been estimated to be 100×10⁹ tons per year worldwide, and half of that is contained in cellulose. If this material, or at least a significant part of it, could be converted into liquid fuel, gas and feed protein, this would constitute a significant contribution to solving the problem of recycling and conservation of resources. The effective utilization of cellulose through biological processes is one approach to overcoming the shortage of foods, feeds, and fuels. However, it has been difficult to develop an economically viable process of converting cellulosic material into fermentable sugars.

The most promising processes involve the use of enzymes which degrade cellulose. These enzymes are collectively known as cellulases and are produced by a number of microorganisms, including fungi (e.g. Trichoderma reseei, Humicola insolens, Fusarium oxysporum) and bacteria (e.g. Clostridium thermocellum, Cellulomonas spp., Thermonospora spp., Bacterioides spp., Microbispora bispora). The economics of the production of fermentable sugars from biomass by means of such enzymes is not yet competitive.

Cellulases from aerobic fungi have received more study than have those of any other physiological group. The filamentous fungus, Trichoderma reesei, is an efficient producer of cellulase enzymes. As such, Trichoderma reesei has been exploited for its ability to produce these enzymes, which are valuable in the production of commodities such as textile and detergents. The cellulolytic mix of Trichoderma reesei is among the best characterized cellulolytic pathways of microorganisms. Three major types of enzymatic activities are found: exoglucanases or exocellobiohydrolases (CBH; CBHI/Cel7A, and CBHII/Cel6A), endoglucanses (EG; EGI/Cel7B, EGII/Cel5A, EGIII/Cel12A, EGIV/Ce61A, and EGV/Cel45A) and β-glucosidases (BG; BGLI/Cel3A and BGLII/Cel1A). These three different types of cellulase enzymes act synergistically to convert cellulose to glucose. The four most abundant components of T. reesei cellulase CBHI/Cel7A, CBHII/Cel6A, EGI/Cel7B, and EGII/Cel5A together constitute greater than 50% of the protein produced by the cell under inducing conditions and can be secreted in excess of 40 g/liter

Cellulases are distinguished from other glycoside hydrolases by their ability to hydrolyze β-1,4-glucosidic bonds between glucosyl residues. The enzymatic breakage of the β-1,4-glucosidic bonds in cellulose proceeds through an acid hydrolysis mechanism, using a proton donor and nucleophile or base. A general feature of most cellulases is a modular structure often including both catalytic and carbohydrate-binding modules (CBMs). The CBM effects binding to the cellulose surface, presumably to facilitate cellulose hydrolysis by bringing the catalytic domain in close proximity to the substrate, insoluble cellulose.

The regulation of cellulolytic enzyme expression in T. reesei is complex and only partially understood. Transcription of the major components of cellulase (CBHI/Cel7A, CBHII/Cel6A, EGI/Cel7B, EGII/Cel5A, EGIII/Cel12A, EGIV/Cel61A, and EGV/Cel45A) is induced not only by cellulose but also by a variety of disaccharides including lactose, cellobiose, and sophorose (glycosyl β-1,2-glucose). Induction by these molecules is antagonized by the presence of the preferred carbon sources, glucose and fructose. Sophorose is by far the most potent inducer of cellulase expression.

Commercial scale production of enzymes is by either solid or submerged culture including batch, fed batch, and continuous flow processes. A problematic and expensive aspect of industrial cellulase production is providing the appropriate inducer to Trichoderma. As is the case for laboratory scale experiments, cellulase production on a commercial scale is induced by growing the fungus on solid cellulose or by culturing the organism in the presence of a disaccharide inducer such as lactose; glucose and/or sophorose are alternative inducers. Unfortunately on an industrial scale, both methods of induction have drawbacks which result in high costs being associated with cellulase production.

The production of cellulase is subject to both cellulose induction and glucose repression. Thus, a critical factor influencing the yield of cellulase enzymes or heterologous proteins under the control of an inducible promoter and/or temperature sensitive promoter (e.g., the cbh1 promoter) is the maintenance of a proper balance between cellulose substrate and glucose concentration. This balance between induction by cellulose and repression by glucose is critical for obtaining reasonable commercial yields of cellulase enzyme. Although cellulose is an effective and inexpensive inducer, controlling the glucose concentration when Trichoderma is grown on solid cellulose can be problematic. At low concentrations of cellulose, glucose production may be too slow to meet the metabolic needs of active cell growth and function. On the other hand, cellulase synthesis can be halted by glucose repression when glucose generation is faster than consumption. Thus, expensive process control schemes are required to provide slow substrate addition and monitoring of glucose concentration (Ju and Afolabi, Biotechnol. Prog., 91-97, 1999).

Presently, due to the critical importance of cellulase enzyme in the process for generating biofuels, a need clearly exists for novel methods to increase cellulase production from filamentous ascomycete fungi, e.g., Trichoderma reesei such that the cellulase enzyme can be economically available to the alternative fuel industry for their endevours to provide technology which would reduce dependency on oil. For such industrial application, highly efficient expression systems must be available that produce higher yields of cellulase proteins. Reducing the cost of cellulose enzyme production is a key issue in the enzymatic hydrolysis of lignocellulosic materials. Specifically, the need exists for the obtaining and isolating of improved fungal strains capable of increased production of cellulase enzymes. Obtaining and isolating improved strains that produce cellulase at higher temperatures also has advantages as the fermentation can withstand fluctuations in temperature without product loss, and cooling costs are reduced. The present invention addresses these needs by providing methods for selecting the highly desired productive fungal strains.

These enzyme activities have many uses in textile, food and animal feed, detergents, pulp and paper industries, and for fuel ethanol production.

### 4. SUMMARY OF THE INVENTION

The present invention relates to methods for selecting improved filamentous fungal strains for enzyme production. In one embodiment, the enzyme is selected from the group consisting of glucoamylases, amylases, cellulases and xylanases. In a further embodiment the enzyme is cellulases. The present invention provides a flexible collection of selection techniques that can be applied in various combination and iteration. Depending on the objectives and scale of the strain improvement project, different methods based on the invention can be devised.

In one embodiment, the method comprises generating genetic diversity in a strain of filamentous fungus thereby producing a population of genetically diverse test cells; selecting the population of test cells in a medium comprising cellulose which is the sole source of carbon and energy and at a temperature that inhibits production of a cellulase enzyme by the parental cells; and isolating cells that overproduce the cellulase enzyme. In another embodiment, the method comprises contacting parental cells of a strain of filamentous fungus with a mutagen thereby producing a population of test cells; selecting the test cells with at least one of the following steps: culturing the test cells at a temperature that inhibits production of a cellulase enzyme by the parental cells; or culturing the test cells in a medium comprising cellulose which is the sole source of carbon and energy in the medium and isolating improved cells that produce more cellulase enzyme than that produced by the parental cells. In a related embodiment of the invention, both selection techniques are performed in the method sequentially and simultaneously. In a specific embodiment, the agent that generates genetic diversity in the fungal cells is not a cytogenetic agent, such as colchicine.

In various embodiments, the selection step is reiterated at least once. In particular, at least one of the reiterated selection step comprises culturing the population of test cells at a temperature different from the initial or previous selection step. Alternatively, at least one of the reiterated selection step comprises culturing the population of test cells in a medium comprising cellulose that is at a concentration different from the initial or previous step, or cellulose that is obtained from a different source.

In one embodiment, the isolating step of the invention encompasses plating out the test cells on a solid medium comprising cellulose, and retrieving cells that exhibit growth rates higher than that of the majority of test cells in the population. Furthermore, the solid medium may comprises two layers, wherein only the bottom layer comprises the test cells that grows into the top layer. The fastest growing fungal cells emerge on the surface thereby facilitating its identification and isolation. In various embodiments, the cellulose used in the medium is purified microcrystalline cellulose. In various embodiments, the temperature used in the selection step can be 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., or 48° C. In various embodiments, the strain of filamentous fungus is a strain of a Trichoderma species or a Hypocrea species, preferably Trichoderma reesei, most preferably a strain that is already a high producer of a cellulase enzyme.

### 5. DESCRIPTION OF THE FIGURES

FIG. 1 is a bar graph of total protein production of DE mutants in shake flasks at 28° C., the optimal temperature for protein production by T. reesei. Mutants DE1, DE 11, DE 13 and DE 19 produce significantly more total protein compared to the parent/control strain. The protein production of DE strains is compared to the parent strain 008 (control) at 28° C.

FIG. 2 is a bar graph of total protein concentration and % PCV measurements of DE 1, 13, and 19 using 008 as a control. The graph shows that DE1, 13, and 19 produce more total protein per unit of PCV than the control. Protein production and growth in shake flasks produced by the parent strain 008, and DE1, DE 13 and DE 19. [symbol] =% PCV

FIG. 3 is a bar graph of total protein production of DE mutants in shake flasks incubated at 32° C., a temperature which is detrimental to total protein production in T. reesei. DE strains 1, 3, 6, 7, 8, 9, 10, 12, 13, 14, 16, 17, 18, 19, and 27 showed improvements in protein production at 32° C. DE 6 and DE 12 were selected for further temperature studies. Protein production of DE strains compared to the parent strain 008 (control) at 32° C.

FIG. 4 is a bar graph showing total protein yield of DE6, DE8 of DE 6 and DE 12 at temperatures 28° C., 32° C., and 34° C. compared to the parent strain 008. FIG. 4 shows that protein production of the control, 008, decreases more than 50% between 28° C. and 32° C. to 34° C. Protein production by DE 6 and DE 12 at 28° C. compared to 32° C. and 34° C. decreases by about 10-15% and 20-25%, respectively. DE 6 and DE 12 are capable of producing more total protein at higher temperatures than the parent strain 008. *=% PCV

### 6. DETAILED DESCRIPTION OF THE INVENTION

The present invention provides a system of selection techniques for selecting and identifying a novel fungal strain that produces an amount of cellulase enzymes that is greater than a parental strain. The system is highly valuable to the development of improved fungal species and strains. The present invention also provides a system of selection techniques for selecting and identifying a novel fungal strain that produces an amount of cellulase enzymes that is greater than a parental strain at temperatures above 28° C.

The very large quantity of biomass-degrading enzymes synthesized by saprophytic fungi, such as Trichoderma reesei, requires a significant investment of cellular resources. Evidence suggests that a primary means by which the cell manages these demands is to regulate transcription of the genes encoding these enzymes according to the availability of different carbon sources. It is unclear to what extent each cellulase enzyme-encoding gene has a unique regulatory apparatus and to what extent expression of these genes is coupled among themselves and with the hemicellulases and the respective secretion mechanism via sharing of regulatory pathways. Few of these pathways have been systematically investigated. It has been speculated that in nature low levels of constitutively expressed cellulases, perhaps in conjunction with a β-glucosidase, must exist to initiate cellulose digestion and generate smaller inducing molecules. These molecules, such as cellobiose and potentially sophorose (generated by trans-glycosylation), would then mediate the induction of the full complement of cellulase-encoding genes. However, data in support of such a hypothesis do not exist for the majority of fungal strains that are of interest.

Rather than using a reductionist approach to understand the regulatory mechanisms and then design novel fungal strains for testing, the inventor devised a set of techniques that can be used individually or in combination to select and identify fungal isolates that produces elevated levels of cellulase enzymes; in particular, at the optimal production temperature of the parent or at higher temperatures. The methods of the invention involve generating genetic diversity in the fungal cells, growing the population of genetically diverse fungal cells under various conditions that are adverse to cellulase production, and selecting isolates that can produce cellulases. Isolates that are capable of producing cellulases under such conditions are expected to have improved yield and/or desirable characteristics for large-scale manufacturing of cellulases.

Trichoderma reesei produces cellulases optimally below 28° C. At temperatures above 28° C., cellulase production is reduced. Strict control of the temperature during the fermentation can be problematic leading to reduced productivities of the cellulase producing strains. Furthermore, cooling the fermentations can be costly.

Many industrially important fungal strains that produce cellulases can be subjected to the system of selection techniques provided in the invention. The methods can also be applied to fungal strains that are genetically engineered to be a high producer of cellulase enzymes or that produces one or more recombinant cellulase enzymes. Details of the fungal host strains that can be used with the invention are described in Section 6.1.

In one embodiment, the invention involves a selection technique that exploits the use of pure cellulose as the sole source of carbon and energy. In another embodiment, the invention provides a selection technique that relies on growing the population of genetically diverse fungal cells at temperatures that are normally inhibitory to cellulase production. In a further embodiment, the selection methods described herein rely on growing the population of genetically diverse fungal cells on a substrate that is difficult to metabolize as a sole carbon source. Such substrates include, but are not limited to, retrograde starch, microcrystalline cellulose and/or xylan. Details on these selection conditions and associated culture techniques are discussed in Section 6.2 herein below.

As used herein the term “parental strain” refers to the strain of fungal cells that exist prior to exposure to an agent that generates genetic diversity according to the invention. Preferably, the parental strain is already a highly productive strain or possesses favorable characteristics. After exposure to the agent, the “parental cells” becomes a population of genetically diverse test cells. Due to the random nature of the process for generating genetic diversity, many different types of cells are expected to be generated. Therefore, when the selection techniques are applied, a large number of different test cells are placed under selection. The term “test cells” as used herein refers to genetically diverse fungal cells generated from a parent strain of filamentous fungus according to the invention. The term also encompasses any progeny of the test cell. “Improved cells” are isolated for their growth characteristics and/or level of enzyme production as determined in the assays of the invention. The term also encompasses any progeny of the improved cell. The improved cell(s) can be isolated and propagated to establish a new improved fungal strain. Non-limiting examples of improved cells exhibit the following characteristics: (i) a higher production level of one or more enzymes at normal temperature; (ii) the ability to maintain a level of enzyme production that is equal or better than the parental cells at elevated temperatures; and/or (iii) an overall improved yield determined, for example, by measuring gram of enzyme produced per gram of carbon input.

According to the invention, starting with a parental strain, the selection techniques in the system can be applied sequentially for one or more rounds of selection. The techniques can be repeated and applied in different order and on different scales depending on the scope of the experiment. The combination of selection techniques and their applications are described in details in Section 6.3.

The system provides the generation of genetically diverse cells from a strain of filamentous fungus, selecting the cells with desirable properties, and reiterating either the selection step(s) or the mutagenesis and selection cycle in order to generate further improved cells. The result is the systematic improvement and accumulation of traits in fungal strains that are desirable for industrial manufacturing of fungal enzymes selected from the group consisting of glucoamylases, amylases, cellulases and xylanases. In one embodiment, any heterologous enzyme under the control of an inducible promoter is contemplated. An example of an inducible promoter is the cbh1 promoter from Trichoderma.

For clarity of disclosure, and not by way of limitation, the detailed description of the invention is divided into the subsections which follow.

### 6.1. Fungal Strains

Many Ascomycetes, Basidiomycetes, and Deuteromycetes are known for their cellulolytic enzymes and/or wood-degrading capability. These fungal species that produce cellulases can be used in the methods of the invention to identify new strains with improved production characteristics. Exemplary genera of fungi that can be targeted for improvement by the methods of the invention include but are not limited to Bulgaria, Chaetomium, and Helotium (Ascomycetes); Coriolus, Phanerochaete, Poria, Schizophyllum and Serpula (Basidiomycetes); and Aspergillus, Cladosporium, Fusarium, Geotrichum, Myrothecium, Paecilomyces, Penicillium, and Trichoderma (Deuteromycetes).

One of the most studied groups comprises species of Trichoderma. The term “Trichoderma” or “Trichoderma species” used herein refers to any fungal organisms which have previously been classified as a Trichoderma species or strain, or which are currently classified as a Trichoderma species or strain, or as a Hypocrea species or strain. Preferably the species are Trichoderma longibrachiatum, Trichoderma reesei, Trichoderma viride, or Hypocrea jecorina. Also contemplated for use as an original strain are cellulase-overproducing strains such as T. longibrachiatum/reesei RL-P37 (Sheir-Neiss et al., Appl. Microbiol. Biotechnology, 20 (1984) pp. 46-53; Montenecourt B. S., Can., 1-20, 1987). In one embodiment, Rut-C30 strain is used. Preferably, the production of cellulases in the species targeted for improvement is tightly regulated and is sensitive to various environmental conditions.

The terms “cellulolytic enzymes” or “cellulase enzymes” refer to (i) endoglucanases (EG) or 1,4-β-d-glucan-4-glucanohydrolases (EC 3.2.1.4), (ii) exoglucanases, including 1,4-β-d-glucan glucanohydrolases (also known as cellodextrinases) (EC 3.2.1.74) and 1,4-β-d-glucan cellobiohydrolases (cellobiohydrolases, CBH) (EC 3.2.1.91), and (iii) β-glucosidases (BG) or β-glucoside glucohydrolases (EC 3.2.1.21). The increase in production and/or secretion of any one or more of these enzymes by a strain of fungal cells is sought in the present invention.

The term “cellulase core activity” refers herein to an amino acid sequence of a truncated cellulase comprising the core domain of the cellobiohydrolases and endoglucanases, for example, EGI, EGII, EGIII, EGV, CBHI or CBHII or a derivative thereof that is capable of enzymatically cleaving a cellulosic polymer.

In yet a further aspect, the present invention relates to a method of producing a truncated cellulase or derivative thereof which method comprises cultivating a host cell as described above under conditions such that production of the truncated cellulase or derivative thereof is effected and recovering the truncated cellulase or derivative from the cells or culture medium.

Highly enriched truncated cellulases are prepared in the present invention by genetically modifying microorganisms described in further detail below. Transformed microorganism cultures are grown to stationary phase, filtered to remove the cells and the remaining supernatant is concentrated by ultrafiltration to obtain a truncated cellulase or a derivative thereof.

In a particular aspect of the above method, the medium used to cultivate the transformed host cells may be any medium suitable for cellulase production in Trichoderma. The truncated cellulases or derivatives thereof are recovered from the medium by conventional techniques including separations of the cells from the medium by centrifugation, or filtration, precipitation of the proteins in the supernatant or filtrate with salt, for example, ammonium sulphate, followed by chromatography procedures such as ion exchange chromatography, affinity chromatography and the like.

Alternatively, the final protein product may be isolated and purified by binding to a polysaccharide substrate or antibody matrix. The antibodies (polyclonal or monoclonal) may be raised against cellulase core or binding domain peptides, or synthetic peptides may be prepared from portions of the core domain or binding domain and used to raise polyclonal antibodies.

The activity of the truncated catalytic core proteins or derivatives thereof as defined herein may be determined by methods well known in the art. (See Wood, T. M. et al in Methods in Enzymology, Vol. 160, Editors: Wood, W. A. and Kellogg, S. T., Academic Press, pp. 87-116, 1988) For example, such activities can be determined by hydrolysis of phosphoric acid-swollen cellulose and/or soluble oligosaccharides followed by quantification of the reducing sugars released. In this case the soluble sugar products, released by the action of CBH or EG catalytic domains or derivatives thereof, can be detected by HPLC analysis or by use of calorimetric assays for measuring reducing sugars.

Other methods well known in the art that measure cellulase catalytic and/or binding activity via the physical or chemical properties of particular treated substrates may also be suitable in the present invention. For example, for methods that measure physical properties of a treated substrate, the substrate is analyzed for modification of shape, texture, surface, or structural properties, modification of the “wet” ability, e.g. substrates ability to absorb water, or modification of swelling. Other parameters which may determine activity include the measuring of the change in the chemical properties of treated solid substrates. For example, the diffusion properties of dyes or chemicals may be examined after treatment of solid substrate with the truncated cellulase binding protein or derivatives thereof described in the present invention. Appropriate substrates for evaluating activity include Avicel, rayon, pulp fibers, cotton or ramie fibers, paper, kraft or ground wood pulp, for example. (See also Wood, T. M. et al in “Methods in Enzymology”, Vol. 160, Editors: Wood, W. A. and Kellogg, S. T., Academic Press, pp. 87-116, 1988).

### 6.2 Selection Methods

The present invention provides a system for selecting a desired strain of filamentous fungus that produces high levels of one or more cellulase enzymes. The system comprises one or more genetic diversification steps and culturing steps conducted sequentially, wherein at each culturing step, the test cells are subjected to selection.

As described earlier, in view of the speculative nature of our knowledge about the induction mechanisms for cellulase production, the design of the selection methods of the invention may appear counterintuitive. In a laboratory setting, where highly purified chemicals are used to grow the fungal strains, it is not a priori certain that the test fungal cells can survive on purified cellulose as the sole carbon and energy source, let alone growing sufficiently to enable its identification and isolation. Furthermore, assuming that a small amount of cellulase enzyme is secreted by one of the mutant cells, it is expected that the secreted enzyme will begin digesting the cellulose and generate enough sugars to trigger a chain reaction of cellulase induction resulting in a burst of growth of fungal cells. Because of the diffusion of sugars in the media (especially in liquid media) and the burst of growth of different mutant cells, other fungal cells in the population can overgrow and mask the mutant fungal cells with the desired phenotypes.

The invention provides a step wherein parental cells or test cells are exposed to an agent that generates genetic diversity in the genome of the cells. In one embodiment, the agent that generates genetic diversity in the methods of the invention is a mutagen that causes localized nucleotide change(s) in the genome. Parental cells and test cells may be mutagenized by such mutagens using any methods known in the art. For example, mutagenesis of the cells can be achieved by irradiation, e.g., ultraviolet light, X-ray, or gamma radiation. Alternatively, mutagenesis can be achieved by treatment with chemical mutagens, e.g., nitrous acid, nitrosamines, methyl nitrosoguanidine, ethylmethanesulfonate, and base analogues such as 5-bromouracil. In one embodiment, insertional mutagenesis is used using transposons, restriction enzyme-mediated integration (“REMI”) or Agrobacterium-mediated transformation.

In a separate embodiment, the agent that generates genetic diversity in the parental cells or test cells is a cytogenetic agent that causes gross changes in the genome, generally at the cytogenetic or chromosomal level, such as but not limited to autopolyploid formation, micronuclei formation, polykaryon formation, chromosomal rearrangement, chromosomal reassortment, chromosomal aberration, chromatid loss, large-scale recombination, etc. Many such agents are known, including but not limited to colchicine (commonly used at 0.1% w/v). As used herein, the term “mutagen” does not encompass such cytogenetic agents. In certain embodiments of the invention, the agent that generates genetic diversity in the parental cells or test cells is not a cytogenetic agent.

In a specific embodiment, the mutagen is applied to spores of the parental strain or test strain, and the surviving spores are plated out on a solid medium. The cells are plated out at various cell densities to facilitate growth and identification by visual inspection or other means. In other embodiments, other forms of the fungal organism beside spores can also be used in the genetic diversification step. Preferably, the agent is a mutagen that is applied at a dose that produces a lethality of about 1-99.9%. In various embodiments, the agent is a mutagen that is applied at a dose that produces a lethality of about 50%, about 60%, about 70%, about 80%, about 90%, or about 95%. In one embodiment, the mutagen is nitrosoguanidine (N-methyl-N′-nitro-N-nitrosoguanidine—MNNG) or ethyl methane sulfonate (nitrogen mustard gas). (see, Gerhardt et al. 1994, Methods for general and molecular bacteriology, American Society for microbiology, p. 297-316).

In another embodiment, the invention provides a culturing step wherein the test cells are grown in a medium comprising cellulose, wherein the cellulose is the sole source of carbon and energy and wherein the medium is substantially free of disaccharides and monosaccharides. In a preferred embodiment, the culturing is performed in liquid phase that enables the culturing and selection of a large population of test cells in batches. Preferably, the cellulose is purified cellulose which includes but is not limited to microcrystalline cellulose, such as AVICEL® (FMC Biopolymer, Philadelphia, Pa.). In water, with shear, AVICEL® forms a three-dimensional matrix comprised of insoluble microcrystals that form an extremely stable, thixotropic gel. The invention provides that the cellulose used in the medium can be obtained from different sources. As the fine structure of cellulose obtained from different sources are likely to be different, it is contemplated that the differences can be exploited to select for the enhanced production of one or more specific cellulase enzymes.

Any minimal medium known in the art for culturing filamentous fungi can be used to prepare the medium for use in the culturing step. Shake flask medium was as described in Ilmen et al., 1997, App Environ Microbiol 63, 1298-1306, except that 100 mM piperazine-N,N′-bis(2-ethanesulfonic acid) (PIPES; Calbiochem) was included to maintain the pH at 5.5. The concentration of cellulose in the medium was about 0.1 to about 2%.

In yet another embodiment, the invention provides a culturing step wherein the test cells or parental cells are grown at a selection temperature that is inhibitive to the production of a cellulase enzyme of interest. Generally, the selection temperature is higher than the temperature at which the test cells or parental cells produce the cellulase enzyme efficiently. In a preferred embodiment, the culturing is performed in liquid phase that enables the culturing and selection of a large population of test cells in batches. At various selection temperatures, the inhibition can limit the production of the cellulase enzyme(s) to about 10%, 20%, 30%, 40%, 50%, 60%, 70%, 80%, 90%, up to about 95%, or 100% of the original level produced by the parental cells or test cells at normal temperature. The selection temperature can be at least 1° C., 2° C., 3° C., 4° C., 5° C., 6° C., 7° C., 8° C., 9° C., 10° C., 11° C., 12° C., 13° C., 14° C., 15° C., or 20° C. higher than a temperature within a temperature range at which cellulase production by the test cells or its parental strain normally occurs. This temperature can be the temperature at which the median level of cellulase production occurs. As a non-limiting example, the normal temperature range for the production of cellulases by Trichoderma reesei is 24° C. to 28° C. According to the invention, the selection temperature for Trichoderma reesei can be 25° C., 26° C., 27° C., 28° C., 29° C., 30° C., 31° C., 32° C. 33° C., 34° C., 35° C., 36° C., 37° C., 38° C., 39° C., 40° C., 41° C., 42° C., 43° C., 44° C., 45° C., 46° C., 47° C., or 48° C. In a specific embodiment, the culturing is performed in liquid phase. Depending on the experimental design, a range of selection temperatures can be used. Choice of selection temperatures for the culturing depends on other factors, such as but not limited to growth rate, viability, and the distribution of different types of cellulases that is produced by the test cells.

The invention also provides a culturing step that combines features of the abovementioned selection techniques. In this embodiment, the test cells can be cultured in a media that comprise cellulose which is the sole source of carbon and energy and at a temperature that is higher than the temperature at which the test cells normally produce a cellulase enzyme.

In a related embodiment, the invention further provides that within a culturing step of the invention, optionally, the cell density of the culture can be adjusted periodically by dilution with fresh medium or by transferring a portion of the culture into one or more volumes or batches of fresh medium. This aspect of the invention is particularly applicable when a liquid media is used. The rationale for adjusting the cell density of the culture is to maintain the selection by reducing the concentration of hydrolytic products of cellulose, such as oligosaccharides or monosaccharides, that are generated by the induced cellulases. Another reason for the adjustment is to reduce the chance that other test cells, especially neighboring test cells, overgrow the improved cells in the culture when the selection is relaxed, i.e., when oligosaccharides or monosaccharides start to accumulate in the culture or diffuse to neighboring test cells from the improved cells (cross-feeding). It is contemplated that the cell density of a liquid culture grown, without or with agitation (100-300 rpm), is maintained within the range of 10/mL to 10⁹/mL. In one embodiment, about 1000 colony forming units/mL are present.

The decision to dilute or transfer the culture in the course of a selection step is dictated in part by factors such as the growth rate of the test cells, the medium composition, and the culture temperature. The total time of each selection step, the dilution factor, the transfer factor, the total number of adjustments made within a selection step, and the interval between each adjustment, can also be determined empirically by one of skill in the art. Based on the examples as described below, it is preferable that the frequency of dilutions and/or transfers increases towards the end of the selecting step.

In various embodiments, the duration of the first transfer is about 5 to about 10 days; the duration of the second transfer is about 3 to about 5 days; the duration of the third transfer is about 2 days to about 4 days; the duration of the fifth transfer is about a day to about 2 days; and the duration of the final transfer is about 16 hours to about 24 hours. In one embodiment, the number of transfers is between 3 and 10. In another embodiment, the total transfer time is about 2 to about 4 weeks. These steps enrich the culture medium to contain a majority of improved stains.

The methods of the invention can also incorporate other culturing and selection techniques. In a related embodiment, the test cells can be cultured in a solid phase assay comprising two layers of solid medium, each layer of medium comprising cellulose as the sole source or a limiting source of energy or carbon. The two layers may comprise the same ingredients and even the same concentrations of ingredients, and are preferably prepared in a plate. The solid media preferably comprises agar, e.g., 1.5% (w/v) agar. The bottom layer comprises a population of test cells, preferably spores, while the top layer does not comprise any fungal cells. The plate comprising a top layer and the fungal cells in the bottom layer are incubated at a temperature for a period of time for the fungal cells to grow within the solid medium. The thickness of the top layer is uniform across the plate and controlled so that the fastest growing fungal cells that consume cellulose emerge at the surface of the top layer. In preferred embodiments, the improved cells are test cells that exhibit growth rates higher than that of the 50^{th}, 60^{th}, 70^{th}, 80^{th}, 90^{th}, 95^{th}, or 98^{th} percentile in growth rate of said population of test cells. The test cells that break the surface of the top layer are visually detectable and can be readily isolated by techniques known in the art. Typically, the thickness of the top layer ranges from about 2.5 mm, 5 mm, 7.5 mm, 10 mm, 12.5 mm, 15 mm, 17.5 mm, 20 mm, 25 mm to about 30 mm.

In various embodiments, this solid phase assay can be used at the end of the selection regime to facilitate isolation of the improved cells and ranking the improved cells by growth rate in cellulose. In a specific embodiment, the solid phase assay can also be carried out at a temperature that is higher than the temperature at which the test cells normally produce a cellulase enzyme.

### 6.3 Methods of the Invention

According to the invention, the selection techniques described in Section 6.2 can be applied individually or in combination in a strain improvement project. In various embodiments, the invention method encompasses at a minimum a genetic diversification step, a selection step and an isolation step. In one embodiment that involves mutagenesis, a starting strain of fungal cells are exposed to a mutagen to generate a genetically diverse population of test cells. The test cells are subjected to selection during culture and the cells that show improvement in the production characteristics for a cellulase enzyme of interest over the parental cells is isolated.

In one embodiment, the method employs a single selection technique selected from either culturing the test cells at a temperature that would inhibit cellulase enzyme production by the parental cells; or culturing the test cells in a medium comprising cellulose which acts as the sole source of carbon and energy in the culture. In another embodiment, the method employs both selection techniques in sequence, i.e., either (i) culturing at a selection temperature followed by culturing in a minimal medium comprising cellulose; or (ii) culturing in a minimal medium comprising cellulose followed by culturing at a selection temperature. In yet another embodiment, both selection techniques are applied simultaneously, i.e., the population of test cells are cultured in a minimal medium comprising cellulose at a selection temperature. In most embodiments, the selection temperature is higher than the normal temperature at which cellulases are produced efficiently.

In various embodiments, each of the selection techniques can be applied multiple times in sequence before the improved cells are isolated. The population of test cells can be cultured at several selection temperatures. For example, the population of test cells can be cultured serially, each successive step at a higher temperature than the preceding step. In a specific embodiment, the test cells are cultured over a period of time when the temperature changes gradually and continuously from the normal temperature to the selection temperature. The population of test cells can be cultured at several different cellulose concentrations or use several different sources of cellulose.

The invention generally encompasses using both selection techniques in sequence and in various order, each technique being applied once or multiple times. For example, a population of test cells can be grown at different selection temperatures before being cultured in a minimal medium comprising cellulose. In another non-limiting example, a population of test cells can be grown at a first selection temperature, then cultured in a minimal medium comprising cellulose, and then cultured at a second selection temperature. In yet another non-limiting example, a population of test cells can be grown in a minimal medium comprising cellulose obtained from a first source, then cultured at a selection temperature, and then cultured in a minimal medium comprising cellulose obtained from a second source. In yet another embodiment, a population of test cells is cultured at various selection temperatures in a minimal medium comprising cellulose.

In various embodiments, the population of test cells under culture may be diluted or transferred serially depending on the cell density and according to the experimental design.

According to the invention, within a strain development project, the selection step can be repeated at least one time. In each of the at least one reiteration, certain aspects of the selection step can optionally be modified. The invention also encompasses a method where the step of generating genetic diversity is also repeated, each to be followed by a selection step. For example, a temperature different from the initial or previous step can be used to culture the test cells; a different concentration of cellulose or a cellulose obtained from a different source may be used in the medium; a different mutagen or a different dose of mutagen may be used to generate diversity.

The present invention may be better understood by reference to the following non-limiting examples, which are provided only as exemplary of the invention. The following examples are presented to more fully illustrate the preferred embodiments of the invention. The examples should in no way be construed, however, as limiting the broader scope of the invention.

### 7. EXAMPLES

### 7.1. Media and Solutions

Minimal Selection Medium was used to select mutant strains with improved properties related to improved total protein production or the ability to produce more total protein at higher temperatures. Minimal Selection Medium was prepared as described by Ilmen et al. 1997, App Environ Microbiol, 63, 1298-1306 expect that the lactose is replaced by 1 g/l AVICEL®.

Cellulase Screening Medium was used in a secondary screen for improved protein production. Cellulase Screening Medium contained 20 ml of 50× Vogels stock solution, 0.5 g of AVICEL® (FMC Biopolymer, Philadelphia, Pa.), and 20 g of Agar, 980 ml of dH₂O. 50× Vogels Stock solution was prepared by dissolving: (1) 150 g of Na₃Citrate.2H₂O; 10 g of MgSO₄.7H₂O; and 5 g of CaCl₂.2H₂O in 300 ml of dH₂O; (2) 250 g of KH₂PO₄ in 500 dH₂O; (3) 100 g of NH₄NO₃ in 200 ml of dH₂O. The two solutions were added together and 5 ml of Vogels Trace Element Solution and 2.5 ml of Vogels Biotin Solution (0.1 g of d-Biotin in 1 liter of dH₂O) was added. Vogels trace elements solution contained 1 liter of dH₂O, 50 g of Citric Acid; 50 g of ZnSO₄.7H₂O; 10 g of Fe(NH₄)₂SO₄.6H₂O; 2.5 g of CuSO₄.5H₂O; 0.5 g of MnSO₄.4H₂O; 0.5 g of H₃BO₃ (Boric Acid); and 0.5 g of NaMoO₄ 2H₂O. (see Davis et al., 1970, Methods in Enzymology 17A, pg 79-143; and Davis et al., 2000, Neurospora, Contributions of a Model Organism, Oxford University Press, for information on Vogels minimal medium).

Total protein production in shake flasks was examined by incubating mutants at 28° C., 150 rpm, for 96 h using 250 ml flasks containing Lactose Minimal Medium as described by Ilmen et al., 1997, App Environ Microbiol 63, 1298-1306. Packed cell volume (PCV) was used to measure growth. Ten mL of broth was collected from a shake flask and placed in a 15 mL, 17×120 mm Sarstedt conical tube (Sarstedt, Newton, N.C.). The tubes were centrifuged at 1500 rpm for exactly 10 minutes and the volume of the pellet was recorded.

### 7.2. Methods

The following experiments began with the Trichoderma reesei strain 008 which is a highly productive strain related to the strain RL-P37. To generate genetic diversity, the cells were mutated with the methylating compound N-methyl-N′-nitro-N-nitrosoguanidine (NTG). NTG is one of the most potent mutagens available; it induces primarily base transition mutations of the GC to AT type (although AT to GC transitions, transversions, and frameshifts arise at low frequencies).

A kill curve was prepared when a strain was mutated for the first time. Starting at time zero, samples were taken every 30 minutes and a viable spore count was conducted. Once the kill curve was established, only the time zero and the final viable count were made to ensure the correct % kill had been obtained. For example, a 50% kill library and a 99% kill library were prepared by incubating the spores with NTG for 1.5 hours, and 3 hours, respectively. After incubation, the NTG was removed by washing the spores at least three times in water. Aliquots were prepared of the mutated spores and they were stored in glycerol at −70° C.

Fresh fungal plates were prepared and used to obtain a spore suspension containing about 1×10⁹ spore forming units/ml. The number of spore forming units/ml was determined using a hemocytometer. A solution of NTG (Aldrich-4991) was freshly prepared to a concentration of 15 mg/mL in DMSO and added at a final concentration of 1.0 mg/ml to the fungal spore suspension. The fungal spore suspension was then incubated at room temperature in the dark until the desired kill level was obtained. In this case, a 99% kill was obtained.

Approximately 1×10⁹ mutated spores were used to inoculate shake flasks that contain Minimal DE Selection Medium and 1 g/l AVICEL®. (see Ilmen et al. 1997, App Environ Microbiol, 63, 1298-1306). The flasks were incubated at 37° C. for 4 weeks with agitation at 150 rpm. During the 4-week period, the culture(s) were serially transferred at increasing frequency.

About 100 μl-500 μl was transferred into 50 ml medium in 250 ml flasks. Transfer was initiated as soon as there were signs of growth by visual appearance of flasks, microscopic examination, and changes in pH. About seven transfers were performed. The first was made after about 10 days, the second after 7 days, the third after 5 days, the fourth after 4 days, the fifth after 2 days, the sixth after 1 day, the last was an overnight culture about 12-16 h old. At the end of the selection step, the culture was sporulated on PDA plates (Difco) at 28° C. Spores were scraped off the plate, resuspended in water and used in a secondary selective screen as described below.

### Isolation of Mutants with Improved Total Protein Production

Cellulase Screening Medium was prepared and cooled to 55° C. in a water bath. In a small petri dish (82 mm), an aliquot containing about one million mutated spores was dispensed in a circle about ½ way between the center of the plate and the edge and 10 ml of the Cellulase Screening Medium (described above) was added. The plate was subsequently swirled so that the spores were dispersed in the middle of the plate, but not dispersed all the way to the edges, and set to harden for about 5-10 minutes. 25 ml of Cellulase Screening Medium was added and allowed to harden. Another 10 ml of Cellulase Screening Medium was then added and the plates were incubated at 28° C. overnight. The next day, the surface of the plate was checked every four hours for growth using a dissecting microscope. For each library, the approximate time that colonies reached the surface of the plate was determined.

The first 1-3 isolates that reached the surface of the agar were collected using a sterile razor blade, ignoring the colonies that came up around the edges. The colony was observed under the microscope and the razor blade was used for touching the surface of the colony, being careful not to dig into the agar. A small piece of mycelia was removed and placed onto PDA and incubated 28° C. Once grown, the isolates were evaluated for total protein production in shake flasks.

One strain, DE1, produced 15% more total protein than the parent strain in shake flasks containing medium described by Ilmen et al., 1997, App Environ Microbiol 63, 1298-1306. The level of protein present was assayed using a protein assay from Pierce.

### Isolation of Mutants with Improved Protein Production at Higher Temperatures

In another set of experiments a similar secondary screen to that described above was used except Cellulase Screening Medium containing spores were incubated at 37° C. and fungal cells that emerge to the surface within 1-4 days were isolated. The isolates from this round of screening were subjected a further round of selection. The second round of selection involves growing the isolates separately at 28° C. and 32° C. in the same Cellulase Screening Medium that comprises 1 g/l AVICEL®.

Strains DE6 and DE12 were further characterized by growing in shake flasks containing medium described by Ilmen et al., 1997, App Environ Microbiol 63, 1298-1306 at elevated temperatures. The level of protein present was assayed using a protein assay from Pierce.

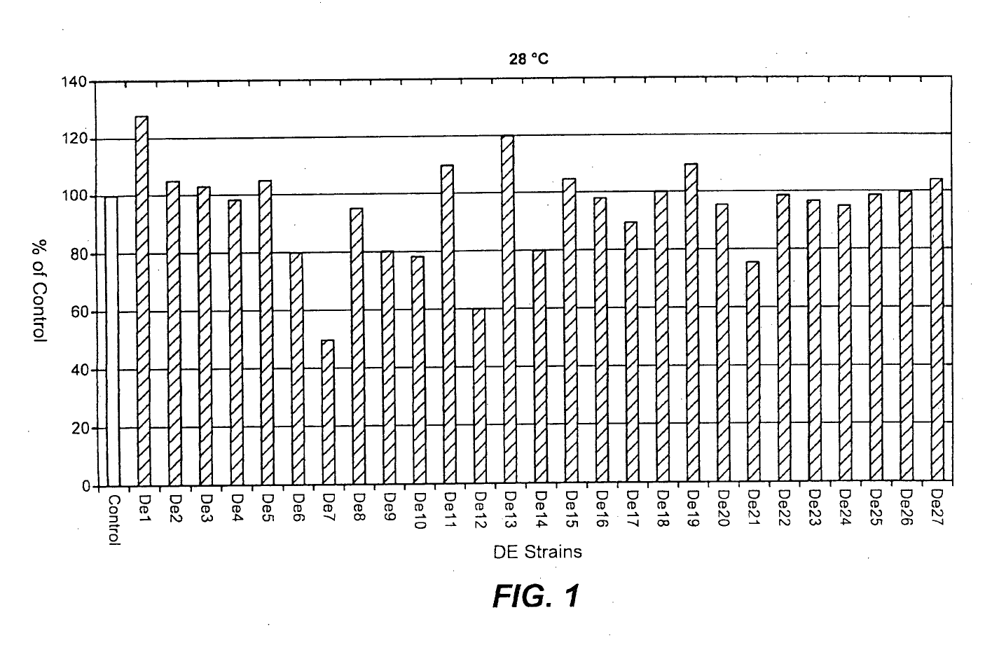

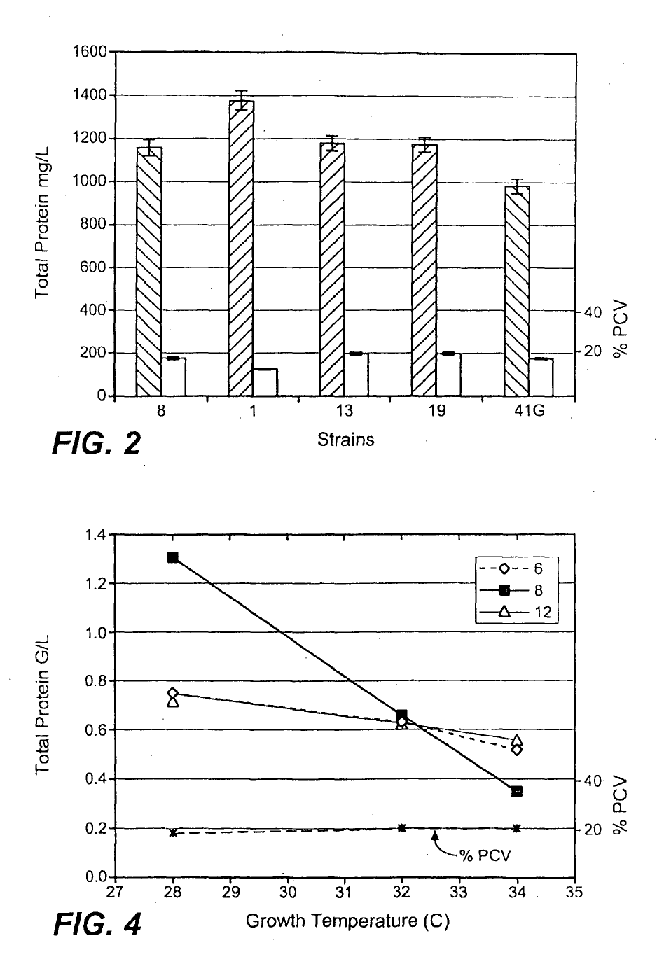

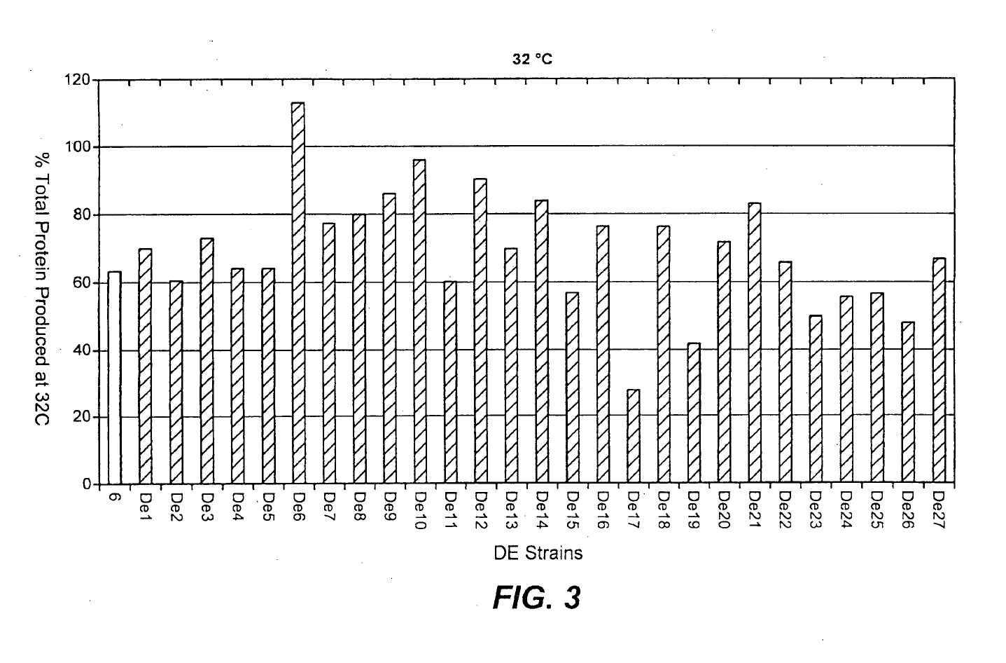

The DE screening method resulted in the isolation of two different types of improved strains. Some mutants showed improvements in total protein production (FIG. 1) while other strains showed the ability to product larger amounts of total protein at higher temperatures than the parent (FIG. 3).

### 8. EQUIVALENTS

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference in its entirety.

What is claimed:

1. A method for obtaining a strain of filamentous fungus that produces a enzyme of interest, said method comprising:
(a) contacting parental cells of a strain of filamentous fungus with an agent that generates genetic diversity thereby producing a population of genetically diverse test cells; and
(b) a selection step comprising culturing said population of test cells in a medium comprising cellulose which is the sole source of carbon and energy and at a temperature that inhibits production of said enzyme by said parental cells; and
(c) isolating improved cells that produce said enzyme at a level that is (i) higher than the level produced by the parental cells or (ii) the same or higher than the level produced by the parental cells at a temperature higher than the optimal production temperature of the parental cells, from said population of test cells.

2. A method for obtaining a strain of filamentous fungus that produces a enzyme of interest, said method comprising:
(a) contacting parental cells of a strain of filamentous fungus with a mutagen thereby producing a population of mutant cells; and
(b) at least one selection step selected from the group consisting of:
(i) culturing said population of mutant cells at a temperature that inhibits production of said enzyme by said parental cells;
(ii) culturing said population of mutant cells in a medium comprising cellulose which is the sole source of carbon and energy in said medium; or
(iii) a combination of (i) and (ii); and
(c) isolating improved cells that produce said enzyme at a level higher than the level produced by the parental cells, from said population of mutant cells.

3. The method of claim 2, said method comprises culturing said population of test cells in a medium comprising cellulose which is the sole source of carbon and energy and at a temperature that inhibits production of said enzyme by said parental cells.

4. The method of claim 2, wherein at least one selection step is carried out in liquid medium which comprises said population of test cells.

5. The method of claim 1 or 2, wherein the selection step is repeated at least one time.

6. The method of claim 5, wherein each at least one repeated selection step comprises culturing said population of test cells at a different temperature.

7. The method of claim 5, wherein each at least one repeated selection step comprises culturing said population of test cells in a medium comprising cellulose that is at a different concentration or that is obtained from a different source.

8. The method of claim 1 or 2, wherein said selection step further comprises adjusting the cell density of the culture at least one time.

9. The method of claim 8, wherein the cell density of the culture is adjusted by transferring a portion of said population of test cells to a volume of fresh medium.

10. The method of claim 1 or 2, wherein said isolating step comprises plating out said population of test cells on a solid medium comprising cellulose, incubating the test cells for a period of time, and retrieving cells that exhibit growth rates higher than that of the 80^{th} percentile in growth rate of said population of test cells.

11. The method of claim 10, wherein said solid medium comprises two layers, wherein only the bottom layer comprises said population of test cells.

12. The method of claim 10, wherein said solid medium comprises two layers, wherein only the bottom layer comprises said population of test cells.

13. The method of claim 1 or 2, wherein the strain of filamentous fungus is a strain of a Trichoderma species.

14. The method of claim 1 or 2, wherein the strain of filamentous fungus is a strain of Trichoderma reesei.

15. The method of claim 1 or 2, wherein the cellulose is purified microcrystalline cellulose.

16. The method of claim 1 or 2, wherein the temperature is selected from the group consisting of 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., and 48° C.

17. The method of claim 1 or 2, wherein said enzyme is selected from glucoamylases, amylases, cellulases, xylanases and any combination thereof.
